(19) United States
(12) Patent Application Publication
(10) Pub. No.: US 2025/0143810 A1
(43) Pub. Date: May 8, 2025

(54) ROBOTIC ASSISTANT FOR ANKLE FRACTURE WITH SYNDESMOTIC INJURY

(71) Applicants: The Johns Hopkins University, Baltimore, MD (US); Children's National Health System, Washingon, DC (US)

(72) Inventors: Babar SHAFIQ, Baltimore, MD (US); Kevin CLEARY, Baltimore, MD (US); Jeffrey H. SIEWERDSEN, Baltimore, MD (US); Wojciech ZBIJEWSKI, Baltimore, MD (US)

(73) Assignees: The Johns Hopkins University, Baltimore, MD (US); Children's National Health System, Washington, DC (US)

(21) Appl. No.: 18/835,603

(22) PCT Filed: Feb. 15, 2023
(86) PCT No.: PCT/US2023/013144

§ 371 Date: Aug. 2, 2024

Related U.S. Application Data
(60) Provisional application No. 63/310,481, filed on Feb. 15, 2022.

(51) Int. Cl. A61B 34/30; A61B 17/00; A61B 17/56; A61B 17/88; A61B 34/20; A61B 90/00; A61G 13/12

(52) U.S. Cl. CPC A61B 34/30; A61B 17/8866; A61B 34/20; A61G 13/125; A61B 2017/0092; A61B 2017/564; A61B 2090/031

(57) ABSTRACT

A robotic system to assist a surgeon during ankle fracture procedures includes an imaging system configured to be at least one of mounted on or arranged adjacent to a robotic device. The system includes a passive arm, a separate actuatable section, and a controller configured to communicate with the actuatable section. The passive arm is structured to be placed on a side of a patient's leg fixed to a platform and the passive arm comprises a fastening mechanism structured to be attached to a tibia of the patient's leg. The actuatable section is structured to be placed on a side of said patient's leg fixed to the platform and the actuatable section comprises a fastening mechanism structured to be attached to a fibula of the patient's leg.

### CROSS-REFERENCE TO RELATED APPLICATIONS

The present patent application claims priority benefit to U.S. Provisional Patent Application No. 63/310,481, filed on Feb. 15, 2022, the entire content of which is incorporated herein by reference. All references cited anywhere in this specification, including the Background and Detailed Description sections, are incorporated by reference as if each had been individually incorporated.

### BACKGROUND

### 1. Technical Field

The currently claimed embodiments of the present invention relate to robots, and more particularly to robotic devices to assist with surgery for ankle fractures.

### 2. Discussion of Related Art

Trauma to the ankle is one of the most common injuries with an incidence of over 2 million ankle injuries reported each year in the United States alone [1]. These injuries could be as simple as a minor sprain to the ankle or so severe that patients come in with extreme bruising, swelling, deformity and an inability to bear weight on the affected limb. Over half a million ankle injuries require surgeries or proper clinical interventions for treatment [2]. In particular, high fibular fracture with syndesmosis disruption account for over 100,000 per year and can result in chronic functional impairment and mechanical instability, which requires a long-term rehabilitation to reverse the effects of muscle atrophy, stiffness, and pain [3,4].

The ankle syndesmosis presents a complex spatial interrelationship of the distal tibia and fibula. Syndesmotic injures occur when one or more of the four ligaments between the tibia and fibula are sprained/tom [5]. These four ligaments rigidly attach the fibula to the tibia, preventing widening of the distal tibiofibular space. When sprained or tom, the fibula dissociates from the tibia by creating a gap and disrupting the ankle mortise [6]. The ankle mortise is formed as a constrained space created between the ends of the tibia and fibula when the ligaments are intact. The talus fits in this space creating the ankle joint. In fractures and sprains where the ankle mortise is disrupted, either by ligament sprain alone or by a combination of sprain and fracture, the talus shifts abnormally resulting in abnormal articulation of these bones leading to instability, pain, arthritis and significantly impaired function [7].

One cadaveric study demonstrated that a 2 mm lateral talar shift led to 42% reduction in the talotibial joint contact area [6]. Another study found that fibular displacement (>2 mm shortening or lateral shift, or greater than 5° of external rotation) significantly increased contact forces on the joint, which relates to a malreduction of the distal tibiofibular joint at surgery [8]. If the distal tibiofibular joint and mortise is not accurately reduced and stably fixed, tibiotalar instability results, which puts the patients at a significantly increased risk for posttraumatic arthritis [6,9,10].

For repair, the distal tibiofibular joint must be reduced accurately and fixated surgically using screws and plates to hold the fibula until the ligaments heal and the integrity of the ankle mortise is restored. In the current clinical workflow, the surgeon must manually correct the maleducation seen on the pre-operative CT scan [11]. Intraoperatively, 2D fluoroscopic imaging is used to evaluate the reduction. This method can result in large fluoroscopy exposures to patient and clinician due to repeated imaging during the procedure. In addition, judging accurate reduction of the fibula to the tibia utilizing 2D fluoroscopy can result in significant malreduction as the true 3D orientation of the articulation cannot be visualized precisely.

Despite the prevalence of postoperative syndesmosis disruption, accurate reduction in the intraoperative setting remains a significant challenge. Therefore, there remains a need for new and/or improved devices and systems for accurate reduction in the intraoperative setting.

### SUMMARY

A robotic system to assist a surgeon during ankle fracture procedures according to some embodiments of the current invention includes an interoperative imaging system configured to be at least one of mounted on or arranged adjacent to a robotic device. The system includes a passive arm, a separate actuatable section, and a controller configured to communicate with the actuatable section. The passive arm is structured to be placed on a side of a patient's leg fixed to a platform and the passive arm comprises a fastening mechanism structured to be attached to a tibia of the patient's leg. The actuatable section is structured to be placed on a side of said patient's leg fixed to the platform and the actuatable section comprises a fastening mechanism structured to be attached to a fibula of the patient's leg.

A method of controlling a robotic device for assisting a surgeon during ankle fracture procedures, in which the robotic device includes a passive arm, an actuatable section that is separate from said passive arm, and a controller configured to communicate with said actuatable section, according to an embodiment of the current invention includes providing a maximum force amount and a maximum torque amount to the controller; and providing instructions to the controller for motion of the actuatable section of the robotic device to assist the surgeon to reduce a distal tibiofibular joint of the patient's leg. The motion is limited to the maximum force amount and the maximum torque amount during operation.

A computer-readable medium according to an embodiment of the current invention contains non-transient computer-executable code which when executed causes a controller for a robotic device to assist a surgeon during an ankle fracture procedure. The robotic device includes a passive arm, an actuatable section that is separate from the passive arm, and a controller that is configured to communicate with the actuatable section. The non-transient computer-executable code causes the controller to receive a maximum force amount and a maximum torque amount, and receive instructions for motion of the actuatable section of the robotic device to assist the surgeon to reduce a distal tibiofibular joint of the patient's leg. The motion is limited to the maximum force amount and the maximum torque amount during operation.

### BRIEF DESCRIPTION OF THE DRAWINGS

Embodiments of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

FIG. 1 is a schematic illustration of a robotic system for assisting surgeons during ankle procedures according to an embodiment of the current invention.

FIG. 2 is a schematic illustration of a robotic device for assisting surgeons during ankle procedures according to an embodiment of the current invention.

FIG. 3 shows an experimental setup for testing the design of the robotic device.

FIG. 4 shows mortise views of the ligaments-cut ankle.

FIG. 5 is a table of generated forces and torques based off past experiments.

### DETAILED DESCRIPTION

Some embodiments of the current invention are discussed in detail below. In describing embodiments, specific terminology is employed for the sake of clarity. However, the invention is not intended to be limited to the specific terminology so selected. A person skilled in the relevant art will recognize that other equivalent components can be employed, and other methods developed, without departing from the broad concepts of the present invention. All references cited anywhere in this specification are incorporated by reference as if each had been individually incorporated.

Accordingly, some embodiments of the current invention are directed to robotic system to provide assistance for an accurate reduction of the distal tibiofibular joint with less radiation exposure to the surgical staff. A goal is to utilize the fluoroscopic images acquired during standard practice and enable a low-profile robot, under surgeon guidance, to accurately reduce the distal tibiofibular joint, using the normal contralateral ankle as a patient specific reference.

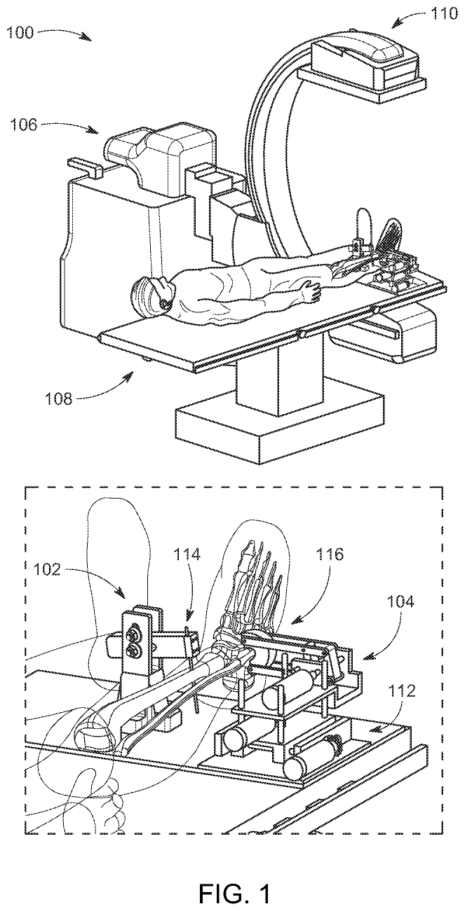

FIG. 1 is a schematic illustration of a robotic system 100 for assisting surgeons during ankle procedures according to an embodiment of the current invention. The robotic system 100 includes a remotely operable imaging system 110 configured to produce fluoroscopic imaging during surgical and orthopedic procedures. The depicted embodiment utilizes fluoroscopy, but other embodiments can utilize other imaging techniques including CT scanning, MRI, Ultrasound, and any other imaging technologies that are used to view the human body in order to diagnose, monitor, or treat medical conditions. The robotic system 100 includes an operation table 108. The robotic system 100 includes a remotely operable actuatable section 104 along with a passive arm 102. Passive arm 102 is structured to be placed on a side of a patient's leg fixed to a platform 112 upon which the patient's leg is to be supported. Passive arm 102 comprises a fastening mechanism 114 structured to be attached to a tibia of the patient's leg. The actuatable section 104 is structured to be placed on a side of said patient's leg fixed to the platform 112 upon which the patient's leg is to be supported. The actuatable section 104 comprises a fastening mechanism 116 structured to be attached to a fibula of said patient's leg. The robotic system 100 also includes a controller 106 that controls the imaging system 110 as well as the actuatable section 104. In some embodiments, the controller 106 is detached from robotic system 100 and can therefore be controlled fully remotely.

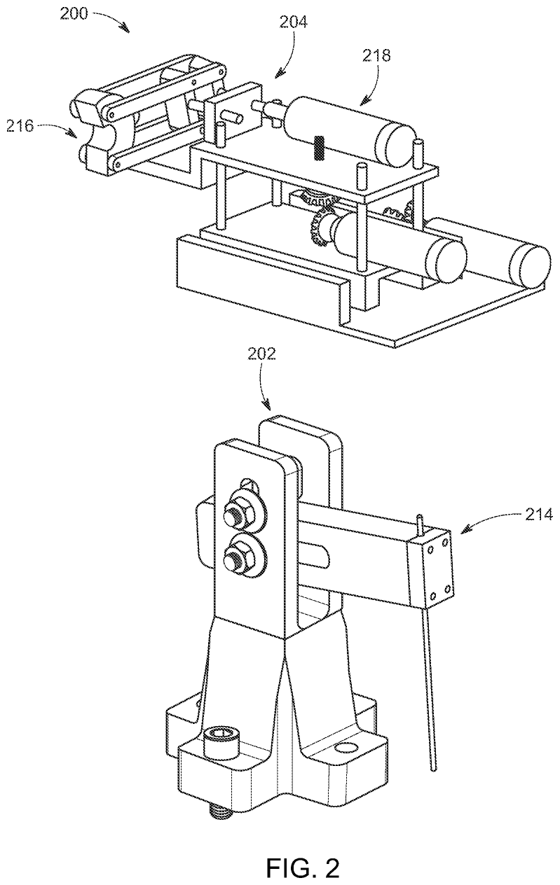

FIG. 2 is a schematic illustration of a robotic device 200 for assisting surgeons during ankle procedures according to an embodiment of the current invention. The robotic device 200 includes a remotely controlled actuatable section 204 and a passive arm 202. The actuatable section 204 and passive arm 202 can be separate from one another or connected via a shared platform (for example, like 112 in FIG. 1). The actuatable section 204 is structured to be placed on the lateral side of a patient's leg. The fastening mechanism 216 of actuatable section 204 comprises a radiolucent end effector 218 that is structured to be attached to said fibula of said patient's leg.

In general, the robotic device 200 can be teleoperated, cooperatively controlled and/or have automated functions or guidance based on intraoperative data registered to preoperative data in various embodiments. For example, in an embodiment, the robot's path can be guided using acquired radiographic images (hence its radiolucent design). The surgeon can have control over this motion at all times, for example through: (1) use of an “emergency-stop” to terminate the motion; (2) actively pushing the robot to a target using cooperative-control; or (3) manually/teleoperatively guiding the robot towards the target established from imaging.

The specific forces needed to perform to reduce the distal tibiofibular joint are not well understood. A study was conducted to quantify the forces associated with reduction of the ankle syndesmosis to help define the requirements for the robotic device 200 design. However, the general concepts of the current invention are not limited by the data only.

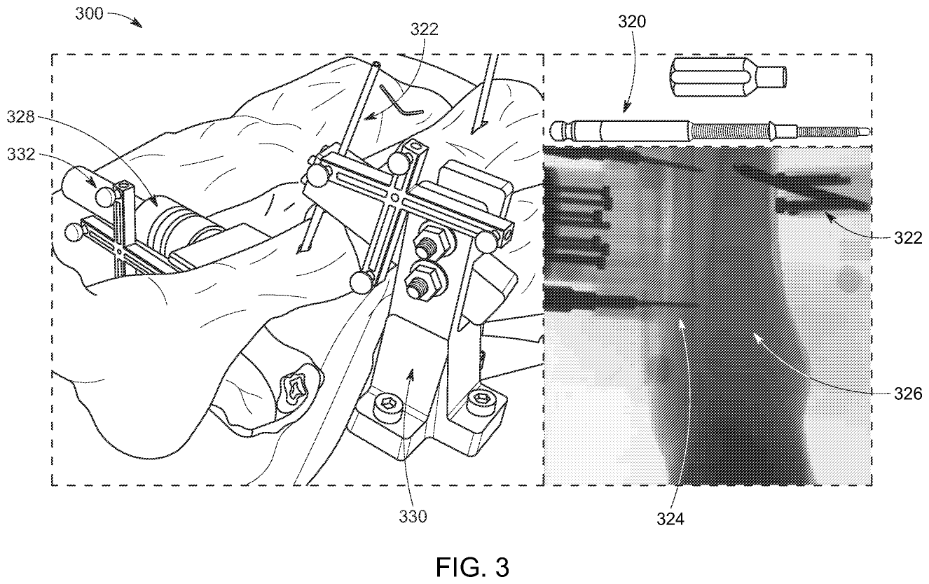

A custom fixture jig 330 component was developed, shown in FIG. 3, made of an onyx plastic with a carbon fiber reinforcement material (Mark Two, Markforged™ 3D printer). These materials are examples for some embodiments and are not intended to limit broader concepts of this invention. The purpose of the fixture 330 is to secure the tibia 326 using a Schanz pin 322 in this example during the reduction and prevent it from moving on the operating table. The fixture can provide adjustment in two degrees of freedom (translational and rotational) to accommodate different attachment locations and orientations. For example, the principal axis of the fibula 324 can be defined as the axis of rotation. The other two translational degrees of freedom can allow us to dynamically (virtually) redefine this axis to accommodate different fibula 324 sizes.

A handheld fibula grasping plate with a force/torque (F/T) transducer 328 (Mini 45, ATI Industrial Automation™, Apex NC) was developed to measure the forces associated with manipulation of the fibula 324. The grasping plate was printed with the same material as the fixture jig 330, and it can be secured to the fibula via two Whirlybird screws 320.

FIG. 3 shows an experimental setup 300 for measuring manipulation forces on a human ankle cadaver. Two separate studies were conducted on a single cadaveric specimen. The first study was performed on an intact ankle without ligament injury and the second study was with the syndesmosis ligaments cut. A below the knee right cadaver leg was obtained from the Maryland Anatomy Board. The cadaver was placed in a supine position on the operating table. As shown in FIG. 3, a Schanz pin 320 was first driven into the tibia 326 and then secured in place using the fixture jig 330 to keep it in position. For the first study, an incision was made to expose the higher fibula region, leaving the syndesmosis ligaments intact. The handheld grasping plate with embedded F/T transducer 328 was then attached to the fibula 324 using two Whirlybird screws 320. The Whirlybird screw 320 is a self-drilling screw with a sleeve used to compresses and fix the grasping plate to the fibula 324. Optical tracking markers 332 were also placed on the fixture jig 330 and the grasping plate to measure the corresponding displacements of the fibula 324 relative to the tibia 326.

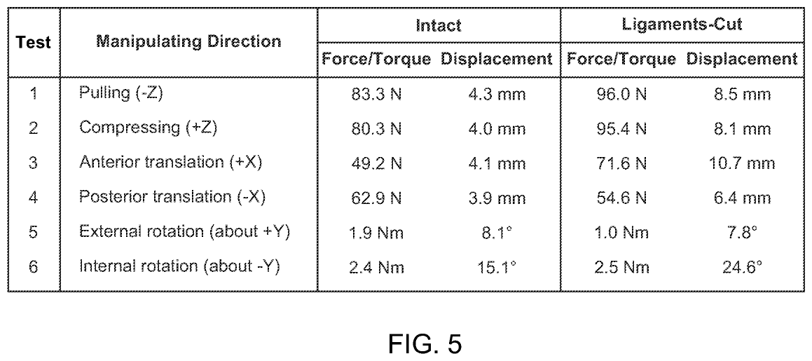

Six fibula manipulation techniques were performed by an orthopedic surgeon on the three principal directions of ankle reduction (i.e., lateral-medial translation, anterior-posterior translation, and external-internal rotation) as summarized in FIG. 5. The manipulation forces and corresponding displacements were recorded using the F/T transducer 328 and optical tracker respectively during the test. Intraoperative fluoroscopic images were also acquired to visualize the anatomy of ankle. The same measurements were then repeated for the second study, after separating the tibia 326 and the fibula 324 through cutting the four syndesmotic ligaments (anterior tibiofibular, posterior tibiofibular, inferior transverse, and interosseous) to mimic a sprain of ankle syndesmosis. Notably, in the setting of a fibula fracture with associated syndesmotic ligament disruption, the fibula will be surgically repaired with a plate and/or screws and then the robot will be employed. The experiments mentioned were done without a fibula fracture simulating the situation of an isolated syndesmotic sprain without fracture or a syndesmosis sprain with fracture (that has already been repaired).

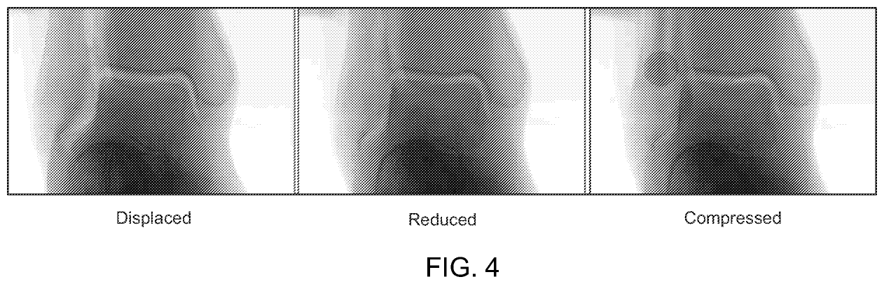

The maximum forces and their respective displacements for each manipulation techniques are reported on FIG. 5. Representative fluoroscopic images of the reduction were demonstrated in FIG. 4. For the purposes of defining the requirements for robotic assistance, the forces and displacements that are of interest are the maximum values on the three principal directions of the reduction.

The results demonstrated the maximum force applied to the lateral direction (Z) to be 96.0 N with maximum displacement of 8.5 mm, applied to the anterior-posterior direction (X) to be 71.6 N with maximum displacement of 10.7 mm, and the maximum torque applied to external-internal rotation (about Y) to be 2.5 Nm with maximum rotation of 24.6°.

The data can be used in: (1) establishing the force/torque requirements for the actuators used and (2) implementing/enforcing safety force limits, for example. In addition, some embodiments can employ limit switches to constrain displacement, for example. As the ranges are small, the motor speed/motion will be slow to provide adequate time for surgeon to react. This speed can be adjustable by the surgeon.

While various embodiments of the present invention have been described above, it should be understood that they have been presented by way of example only, and not limitation. Thus, the breadth and scope of the present invention should not be limited by any of the above-described illustrative embodiments, but should instead be defined only in accordance with the following claims and their equivalents.

The embodiments illustrated and discussed in this specification are intended only to teach those skilled in the art how to make and use the invention. In describing embodiments of the disclosure, specific terminology is employed for the sake of clarity. However, the disclosure is not intended to be limited to the specific terminology so selected. The above-described embodiments of the disclosure may be modified or varied, without departing from the invention, as appreciated by those skilled in the art in light of the above teachings. It is therefore to be understood that, within the scope of the claims and their equivalents, the invention may be practiced otherwise than as specifically described. For example, it is to be understood that the present disclosure contemplates that, to the extent possible, one or more features of any embodiment can be combined with one or more features of any other embodiment.

1. A robotic device to assist a surgeon during ankle fracture procedures, comprising:
a passive arm;
an actuatable section that is separate from said passive arm; and
a controller configured to communicate with said actuatable section,
wherein said passive arm is structured to be placed on a side of a patient's leg fixed to a platform upon which said patient's leg is to be supported,
wherein said passive arm comprises a fastening mechanism structured to be attached to a tibia of said patient's leg,
wherein said actuatable section is structured to be placed on a side of said patient's leg fixed to said platform upon which said patient's leg is to be supported,
wherein said actuatable section comprises a fastening mechanism structured to be attached to a fibula of said patient's leg, and
wherein said controller is configured to provide instructions to said actuatable section to assist said surgeon to reduce a distal tibiofibular joint of said patient's leg.

2. The robotic device according to claim 1, wherein said passive arm is structured to be placed on a medial side of said patient's leg and said fastening mechanism comprises a Schanz pin, and
wherein said actuatable section is structured to be placed on a side of a lateral side of said patient's leg, said fastening mechanism of said actuatable section comprising an end effector that is structured to be attached to said fibula of said patient's leg with Whirlybird screws.

3. The robotic device according to claim 1, wherein said end effector is translucent to x-rays within an energy range of medical imaging devices.

4. The robotic device according to claim 1, wherein said actuatable section comprises a translation assembly and a rotation assembly structured to apply at least one of a linear force and a torque to said fibula relative to said tibia of said patient's leg.

5. The robotic device according to claim 1, wherein said controller is configured to provide instructions to said actuatable section based on at least one of a preoperative plan registered to interoperative data, teleoperative signals, or cooperative control signals.

6. The robotic device according to claim 4, wherein said controller is configured to limit said linear force to a maximum linear force and said torque a maximum torque to prevent damage to said patient's leg.

7. The robotic device according to claim 6, wherein said maximum linear force and said maximum torque are predetermined empirically.

8. A robotic system to assist a surgeon during ankle fracture procedures, comprising: an interoperative imaging system; and a robotic device arranged proximate said interoperative imaging system,
wherein said robotic device comprises:
a passive arm;
an actuatable section that is separate from said passive arm; and
a controller configured to communicate with said actuatable section,
wherein said passive arm is structured to be placed on a side of a patient's leg fixed to a platform upon which said patient's leg is to be supported,
wherein said passive arm comprises a fastening mechanism structured to be attached to a tibia of said patient's leg,
wherein said actuatable section is structured to be placed on a side of said patient's leg fixed to said platform upon which said patient's leg is to be supported,
wherein said actuatable section comprises a fastening mechanism structured to be attached to a fibula of said patient's leg, and
wherein said controller is configured to provide instructions to said actuatable section to assist said surgeon to reduce a distal tibiofibular joint of said patient's leg.

9. The robotic system of claim 8, wherein said interoperative imaging system is one of a fluoroscopy system, a computed tomography (CT) system, a cone beam CT system, a magnetic resonance imaging (MRI) system, or an ultrasound system.

10. The robotic system according to claim 8, wherein said passive arm is structured to be placed on a medial side of said patient's leg and said fastening mechanism comprises a Schanz pin, and
wherein said actuatable section is structured to be placed on a side of a lateral side of said patient's leg, said fastening mechanism of said actuatable section comprising an end effector that is structured to be attached to said fibula of said patient's leg with Whirlybird screws.

11. The robotic system according to claim 8, wherein said end effector is translucent to x-rays within an energy range of medical imaging devices.

12. The robotic system according to claim 8, wherein said actuatable section comprises a translation assembly and a rotation assembly structured to apply at least one of a linear force and a torque to said fibula relative to said tibia of said patient's leg.

13. The robotic system according to claim 8, wherein said controller is configured to provide instructions to said actuatable section based on at least one of a preoperative plan registered to interoperative data, teleoperative signals, or cooperative control signals.

14. The robotic system according to claim 12, wherein said controller is configured to limit said linear force to a maximum linear force and said torque a maximum torque to prevent damage to said patient's leg.

15. The robotic system according to claim 14, wherein said maximum linear force and said maximum torque are predetermined empirically.

16. A method of controlling a robotic device for assisting a surgeon during ankle fracture procedures, said robotic device comprising:
a passive arm;
an actuatable section that is separate from said passive arm; and
a controller configured to communicate with said actuatable section,
said method comprising:
providing a maximum force amount and a maximum torque amount to said controller;
and
providing instructions to said controller for motion of said actuatable section of said robotic device to assist said surgeon to reduce a distal tibiofibular joint of said patient's leg,
wherein said motion is limited to said maximum force amount and said maximum torque amount during operation.

17. The method of claim 16, wherein said providing said maximum force amount and said maximum torque amount is based on empirical data.

18. The method of claim 16, wherein said providing instructions to said controller provides instructions based on at least one of user input for teleoperative control, user input directly to said robotic device by a user based on cooperative control, or a preprogramed task.

19. A computer-readable medium containing non-transient computer-executable code, when executed causes a controller for a robotic device to assist a surgeon during an ankle fracture procedure, said robotic device comprising:
a passive arm;
an actuatable section that is separate from said passive arm; and
a controller configured to communicate with said actuatable section,
wherein said non-transient computer-executable code causes said controller to:
receive a maximum force amount and a maximum torque amount; and
receive instructions for motion of said actuatable section of said robotic device to assist said surgeon to reduce a distal tibiofibular joint of said patient's leg,
wherein said motion is limited to said maximum force amount and said maximum torque amount during operation.

20. The computer-readable medium of claim 19, wherein said maximum force amount and said maximum torque amount are based on empirical data.

21. The computer-readable medium of claim 19, wherein said instructions are based on at least one of user input for teleoperative control, user input directly to said robotic device by a user based on cooperative control, or a preprogramed task.
